(19) United States
(12) Patent Application Publication
(10) Pub. No.: US 2018/0220964 A1
(43) Pub. Date: Aug. 9, 2018

(54) BIOLOGICAL INFORMATION MEASURING INSTRUMENT

(71) Applicants: SHARP KABUSHIKI KAISHA, Sakai City, Osaka (JP); OSAKA PREFECTURE UNIVERSITY PUBLIC CORPORATION, Sakai-shi, Osaka (JP)

(72) Inventors: HIROSHI SAKAYA, Sakai City (JP); TETSUYA HAYASHI, Sakai City (JP); AZUSA NAKANO, Sakai City (JP); SHUNSUKE SHIMAMURA, Sakai-shi (JP); TERUMASA SHIMADA, Sakai-shi (JP)

(73) Assignees: SHARP KABUSHIKI KAISHA, Sakai City, Osaka (JP); OSAKA PREFECTURE UNIVERSITY PUBLIC CORPORATION, Sakai-shi, Osaka (JP); OSAKA PREFECTURE UNIVERSITY PUBLIC CORPORATION, Sakai-shi, Osaka (JP)

(21) Appl. No.: 15/750,952

(22) PCT Filed: Aug. 5, 2016
(86) PCT No.: PCT/JP2016/073195

§ 371 Date: Feb. 7, 2018

(30) Foreign Application Priority Data
Aug. 7, 2015 (JP) 2015-157586

(51) Int. Cl. A61B 5/00; A61B 5/0408; A61B 5/0428

(52) U.S. Cl. CPC A61B 5/6831; A61B 5/0408; A61B 2562/0209; A61B 5/04286; A61B 2503/40; A61B 5/6823

(57) ABSTRACT

A wearable instrument is provided that measures biological information over an extended period of time by enabling an electrode to be readily pressed to an axilla of an animal. The wearable instrument includes: electrodes to be placed under the left and right axillae of a dog to produce an electrocardiogram; pull-up straps to be placed in direct contact with the axilla to press the electrodes onto the axilla; and a cinch strap placed across the anterior chest of the dog to draw the left and right sections of the pull-up strap together.

### TECHNICAL FIELD

The present invention, in one aspect thereof, relates in general to biological information measuring instruments and in particular to biological information measuring instruments that are suited for measurement of biological information of hairy and other like animals.

### BACKGROUND ART

It is widely recognized that everyday health management plays an important role in the prevention and treatment of lifestyle diseases. Accordingly, much attention has been paid to the importance of individuals managing their own health by routinely measuring and recording biological information such as body temperature, body weight, body fat (including visceral fat), bone density, blood pressure, and the amount of physical activity (e.g., number of steps walked and calorie consumption).

The same growing awareness is emerging amongst animal lovers. Owners of companion animals recognize that everyday health management is just as important in the animals as in humans (owners themselves). There is an increasing need for owners to readily measure biological information of their animals.

Patent Literatures 1 and 2 listed below give examples of technology for measuring animals' biological information. Patent Literature 1 discloses a fixture and method for monitoring biological information (e.g., an electrocardiogram) of an animal (e.g., a dog) in a satisfactory manner either without having to trim hair in preparation for medical examination of the site where the animal's biological information is going to be monitored or if preparatory hair trimming is needed, after only trimming the hair to the extent that the animal owner is willing to accept.

Patent Literature 2 discloses a body fat measuring instrument for companion animals, enabling easy and highly precise measurement of animal body fat with minimal inconvenience by impedance technology.

### CITATION LIST

### Patent Literature

Patent Literature 1: Japanese Unexamined Patent Application Publication, Tokukai, No. 2006-141467A (published Jun. 8, 2006)

Patent Literature 2: Japanese Unexamined Patent Application Publication, Tokukai, No. 2005-27661A (published Feb. 3, 2005)

### SUMMARY OF INVENTION

### Technical Problem

The conventional art however has its own issues. The fixture for monitoring biological information of an animal disclosed in Patent Literature 1, although applicable to lengthy electrocardiography of dogs, requires skin hair to be divided as much as possible before biological information monitoring sensors (electrodes) are attached to the anterior chest of a dog (beagle dog) using the M-X lead. The fixture therefore is difficult for unskilled practitioners to attach to the animal in such a manner that the fixture works properly, and it would be safe to say that the fixture has been conceived for use in examinations conducted in dedicated institutions.

The preparation becomes troublesome with dogs that have longer hair than beagles. The electrodes can be attached to dogs with a short hair coat, including the beagle to which the fixture of Patent Literature 1 is attached in a working example described therein, after simply dividing its skin hair. To attach the electrodes to the chest of a dog with a long hair coat or a double coat, however, the dog's chest hair needs to be cut to the extent that it is as short as the skin hair of a beagle.

The body fat measuring instrument for companion animals disclosed in Patent Literature 2 includes a fixture for holding impedance-measuring electrodes pressed onto the skin under the armpits (axillae) of the animal, in the groin (the area where the hind limbs meet the inner parts of the lower abdomen), or on parts of the four limbs that have thin body hair. Dogs and similar animals typically have a hairy chest, but their armpits and groin are less hairy. The animal's hair does not need to be cut or shaved to fit this biological information measuring instrument disclosed in Patent Literature 2 onto the body of the animal

However, the body fat measuring instrument for companion animals disclosed in Patent Literature 2 measures impedance, which can be measured relatively quickly, and is not designed such that the electrodes remain attached to the animal for an extended period of time. Therefore, the electrodes, attached by using the instrument to the armpits, groin, or less hairy sites on the four limbs of a dog, cat, or like companion animal, could be detached or displaced when the animal moves vigorously, lies down, or changes its posture in similar nature, which is a problem with the instrument.

The present invention, in one aspect thereof, is conceived to address these problems, and one of its objects is to provide, for example, a biological information measuring instrument that enables a measuring unit that measures an animal's biological information to be positioned readily and stably under an axilla of the animal.

### Solution to Problem

To address the problems, the present invention, in one aspect thereof, is directed to a biological information measuring instrument to be worn by an animal for measurement of biological information of the animal, the instrument including: at least one measuring unit to be placed under at least one of left and right axillae of the animal to measure the biological information; a first contact section to be placed in direct contact with one of the left and right axillae; a second contact section to be placed in direct contact with the other one of the left and right axillae; a first pull-up section configured to pull up the first contact section toward a dorsal region of the animal; a second pull-up section configured to pull up the second contact section toward the dorsal region; and a cinch section to be placed across an anterior chest of the animal to draw the first and second pull-up sections together, wherein at least one of the first and second contact sections is configured to press the measuring unit to the associated one of the axillae.

### Advantageous Effects of Invention

The present invention, in one aspect thereof, advantageously enables a measuring unit that measures biological information of an animal to be placed readily and stably under an axilla of the animal.

### BRIEF DESCRIPTION OF DRAWINGS

FIG. 1 is a schematic diagram of an exemplary structure of a wearable instrument in accordance with Embodiment 1 of the present invention.

FIG. 2 is an illustration of a dog wearing a securing harness for the electrocardiographic measuring instrument shown in FIG. 1, portions (a), (b), (c), and (d) of FIG. 2 showing the dog as it is viewed from the front, side, above, and below respectively.

FIG. 3 is a schematic diagram of another exemplary structure of a wearable instrument in accordance with Embodiment 1 of the present invention.

FIG. 4 is a schematic diagram of an exemplary structure of a wearable instrument in accordance with Embodiment 2 of the present invention.

FIG. 5 is a schematic diagram of another exemplary structure of a wearable instrument in accordance with Embodiment 2 of the present invention.

FIG. 6 is a perspective view of a typical example of a wearable instrument worn by a dog in accordance with Embodiment 3 of the present invention.

FIG. 7 is a perspective view of an example of the exterior of a storage bag in which the main body of an electrocardiograph is contained.

Portion (a) of FIG. 8 is a diagram representing an exemplary electrocardiographic waveform recorded by the wearable instrument of Embodiment 1 worn by a dog, and (b) of FIG. 8 is a diagram representing an exemplary waveform recorded when an electrode is not correctly pressed onto an axilla of the dog.

FIG. 9 is schematic views of exemplary wearable instruments having different shapes in accordance with an aspect of the present invention.

### DESCRIPTION OF EMBODIMENTS

The following will describe an example of a biological information measuring instrument in accordance with an aspect of the present invention where the invention is applied to a wearable instrument 1 that attaches electrodes to an animal to record electrocardiographic data. The wearable instrument 1 includes two electrodes and a main body 10 (i.e., an electrocardiograph or a biological information processing unit) of an electrocardiograph for recording electrocardiographic data of an animal. The biological information measuring instrument in accordance with an aspect of the present invention is by no means limited to the wearable instrument 1 and may also be used to attach an electrode or a sensor to any one of the left and right axillae of an animal to measure, for example, the body temperature, pulse wave, amount of perspiration, respiration rate, and/or body fat of the animal.

In addition, the following will describe the wearable instrument 1 that secures the electrodes thereof to produce and record an electrocardiogram for a dog. The measurement of biological information is not necessarily performed on dogs and may be performed on various kinds of animals including companion animals such as cats and rabbits, farm animals such as horses and cows, and zoo animals such as lions and chimpanzees.

Companion animals, just like humans, will suffer an arrhythmia and angina unexpectedly irrespective of exercise (physical stress). Neither quick electrocardiography nor stress electrocardiography conducted in general health checks is sufficient to diagnose arrhythmia and angina. It is therefore desirable to, for example, collect electrocardiographic data continuously over 24 or 48 hours (or even longer) in normal health, to discover early symptoms of mild arrhythmia and angina for proper treatment before the condition becomes serious. To make it possible to measure biological information over an extended period, the measuring unit (e.g., electrode) used in the measurement should be such that anyone can readily attach the unit stably to the animal. Additionally, unlike humans, animals will hardly stay still. The measuring unit should, for example, not be displaced by a motion or change of posture of the animal or be capable of, if displaced, readily returning to a suitable position for measurement.

### Embodiment 1

### Structure of Wearable Instrument 1

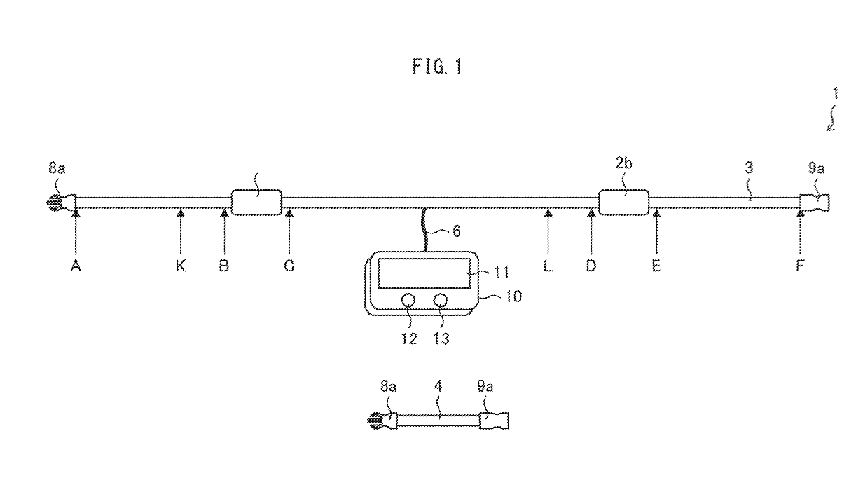

The structure of the wearable instrument 1 in accordance with an embodiment of the present invention will be described in reference to FIG. 1. FIG. 1 is a diagram of an exemplary exterior of the wearable instrument 1. As shown in FIG. 1, the wearable instrument 1 includes an electrode 2a (measuring unit), an electrode 2b (measuring unit), a pull-up strap 3 (first and second contact sections; first and second pull-up sections), and a cinch strap 4 (cinch section).

The pull-up strap 3 is shaped like a strap and intended to be attached onto the body surface of a dog such that the electrodes 2a and 2b are positioned respectively under the left and right axillae of the dog. The pull-up strap 3 therefore has sections that come into direct contact with the dog's axillae (first and second contact sections). The pull-up strap 3 is partly or entirely made of, for example, a stretchy material (e.g., a rubber band). Under a tension (pull-up force) exerted in such a direction that the pull-up strap 3 is elongated between site A and site F, the pull-up strap 3 generates a stress that restores the distance between site A and site F to the original length (a force that presses the electrodes 2a and 2b onto the axillae).

The pull-up strap 3 includes a male end 8a of a buckle (fastening member) and a female end 9a of the buckle (fastening member) for adjusting the length of the pull-up strap 3 when it is worn by the dog and maintaining proper stress exerted on the axillae. The male and female buckle halves 8a and 9a may be of any mechanism that is applicable to a fastening member such as a belt, string, or band and made of any material including metals and plastics. Note that the male and female buckle halves 8a and 8b may be omitted from the structure of the pull-up strap 3. For example, the ends of the pull-up strap 3 may be manually tied by the user. A hook and loop fastener may be alternatively used. Additionally, a length adjuster (not shown) may be used together.

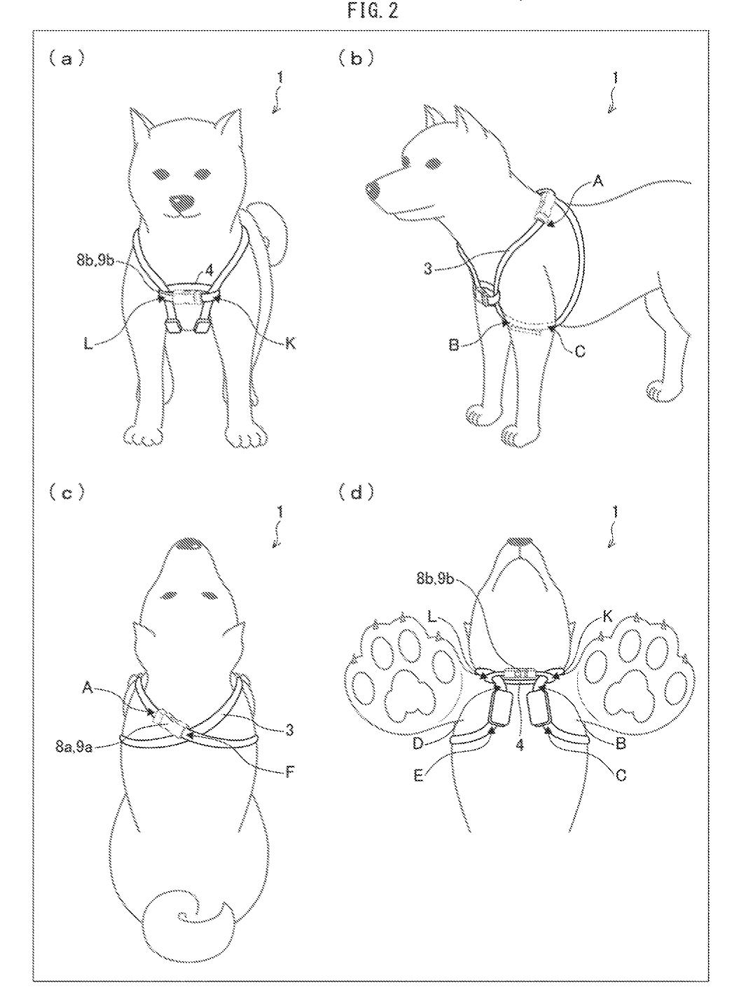

The electrodes 2a and 2b are bipolar electrodes (dielectric electrodes) for measuring an action current caused by excitation of heart muscle (thereby producing measurements). Either the first or second electrode functions also as a ground electrode. The electrodes 2a and 2b are placed across the heart, one on the left axilla and the other on the right axilla. In the wearable instrument 1 shown in FIG. 1, the electrodes 2a and 2b are disposed respectively on portion BC and portion DE of the pull-up strap 3 such that they can be placed under the axillae of the dog (see FIG. 2).

Sponge or like elastic material may be provided between the electrodes 2a and 2b and the pull-up strap 3 so that the electrodes 2a and 2b can fit the axillae snugly when they are pressed onto the axillae by the pull-up strap 3. Alternatively, the electrodes 2a and 2b may be flexible and shaped like a net or comb so that they can deform to the shape of the axillae onto which they are pressed. These arrangements reliably maintains the contact between the electrodes 2a and 2b and the axillae even when the dog is walking or running.

The electrodes 2a and 2b are electrically connected to the main body 10 of the electrocardiograph via a connecting cable 6 (connecting member) containing an electrically conductive substance (e.g., a metal or a conductive organic material). The electrodes 2a and 2b transmit a signal representing measurements of the action current to the electrocardiograph main body 10 that implements Holter monitoring.

The electrodes 2a and 2b may be either wet electrodes that are attached only after a gel is applied or dry electrodes that do not require gel application. Dry electrodes are preferred however when they are attached to the axillae for an extended period, because dry electrodes need no gel application and therefore are less likely to cause skin problems in the axillae.

The wearable instrument 1 may include both the electrodes 2a and 2b or only either one of the electrodes 2a and 2b, depending on the biological information to be measured. For example, the wearable instrument 1 may include a single electrode or sensor that is to be pressed to either the left or right axilla if the body temperature, pulse wave, amount of perspiration, and/or body fat of an animal is to be measured.

The cinch strap 4 is arranged to be positioned across the dog's anterior chest, such that the cinch strap 4 can draw together those sections of the pull-up strap 3 which are run over the left and right parts of the dog's anterior chest. Similarly to the pull-up strap 3, the cinch strap 4 may be partly or entirely made of, for example, a stretchy material (e.g., a rubber band). The cinch strap 4 may also include buckle halves 8b and 9b to maintain the cinch strap 4 drawing the left and right front portions of the pull-up strap 3 together.

The electrocardiograph main body 10 receives an action current caused by excitation of heart muscle from the electrodes 2a and 2b and produces output data representing the dog's electrocardiogram from the action current. The electrocardiograph main body 10 shown in the figure may include a display unit 11 for displaying the output data produced, an ON/OFF button 12 that is operated by the user to start and end the recording of electrocardiographic data, and a red/blue lamp 13 for indicating whether or not either one of the electrodes 2a and 2b is pressed correctly to a corresponding one of the axillae.

### Wearable Instrument 1 as Worn by Dog

Next, referring to FIG. 2, the wearable instrument 1 will be described as it is worn by a dog. FIG. 2 is an illustration of a dog wearing a securing harness for the electrocardiographic measuring instrument shown in FIG. 1. Portions (a), (b), (c), and (d) of FIG. 2 show the dog as it is viewed from the front, side, above, and below respectively. In the example shown in FIG. 2, the pull-up strap 3 has its ends A and F on the back of the dog such that the pull-up strap 3 crosses itself on the back.

Portions of the pull-up strap 3 are in direct contact with the body surface of the dog as detailed below:

Portion AB (first pull-up section, second pull-up section): from the dorsal region to the front part of the axilla of the left forelimb

Portion BC (first contact section, second contact section): the left axilla

Portion CD (first pull-up section, second pull-up section): from the rear part of the axilla of the left forelimb to the front part of the axilla of the right forelimb

Portion DE (first contact section, second contact section): the right axilla

Portion EF (first pull-up section, second pull-up section): from the rear part of the axilla of the right forelimb to the dorsal region

In contrast, the cinch strap 4 draws the left and right front portions of the pull-up strap 3 together across the dog's anterior chest (parallel to portion KL in the figure).

In a manner opposite from the example shown in FIG. 2, the pull-up strap 3 may be worn by the dog such that portion BC is in direct contact with the right axilla and portion DE is in direct contact with the left axilla. Alternatively, the pull-up strap 3 shown in FIG. 1 may be worn by winding twice or more times around the trunk of the dog.

The pull-up strap 3 and the cinch strap 4, as they are shown in FIG. 2, may be described as follows. The pull-up strap 3 presses the electrodes 2a and 2b onto the axillae by pulling up portions BC and DE in a direction from the front part of each axilla of the dog (animal) toward the shoulder or back of the dog (first direction) and in a direction from the rear part of each axilla toward the shoulder or back of the dog (second direction). Meanwhile, the cinch strap 4 draws together, across the dog's anterior chest (third direction), the left and right front portions of the pull-up strap 3 pulling up portions BC and DE in the first direction.

With this arrangement, in the wearable instrument 1, the pull-up strap 3 is continuously pressing the electrodes 2a and 2b to the axillae, and the cinch strap 4 is holding the pull-up strap 3 in place. Therefore, when the dog moves as it likes, the electrodes 2a and 2b, being pressed to the dog's axillae, are not dislocated from the axillae or displaced out of the pressing positions.

It is not difficult at all to attach the wearable instrument 1 to a dog. For example, any animal owner can do it easily. Therefore, by using the wearable instrument 1, the electrodes 2a and 2b are simply and conveniently maintained pressed onto the dog's axillae in a stable manner. Therefore, biological information of a companion animal can be measured by attaching an electrode or like device to the body of the animal without having to cut the body hair of the site where the electrode is to be attached or having to constrain the motion of the body of the animal such that the attached electrode is not displaced while the biological information is being measured.

### Variation Example

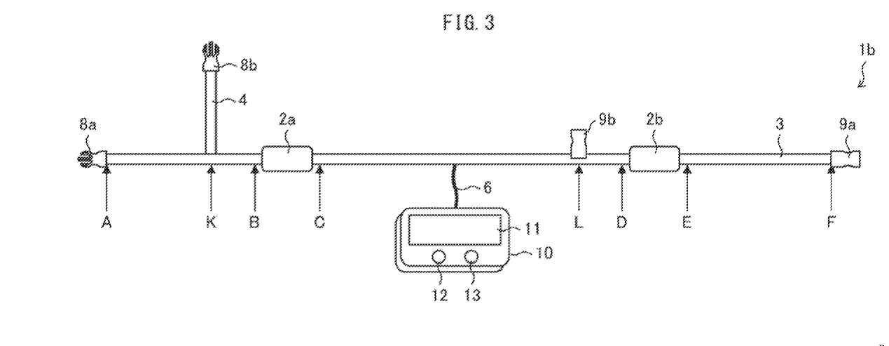

FIG. 1 shows an example where the cinch strap 4 and the pull-up strap 3 are provided as separate members. The embodiment is by no means limited to this arrangement. Alternatively, for example, as shown in FIG. 3, the cinch strap 4 and the pull-up strap 3 may be provided as a single piece. FIG. 3 is a schematic diagram of another exemplary structure of such a wearable instrument 1b.

In the wearable instrument 1b shown in FIG. 3, the cinch strap 4 has one of ends thereof fixed to site K on the pull-up strap 3, thereby forming the cinch strap 4 and the pull-up strap 3 as a single piece. A male end 8b of a buckle is provided on the other end of the cinch section 4 opposite site K. The cinch section 4 draws the left and right front portions of the pull-up strap 3 together across the dog's anterior chest (parallel to portion KL, see FIG. 2) by connecting the male end 8b with a female end 9b of the same buckle provided on site L of the pull-up strap 3.

### Embodiment 2

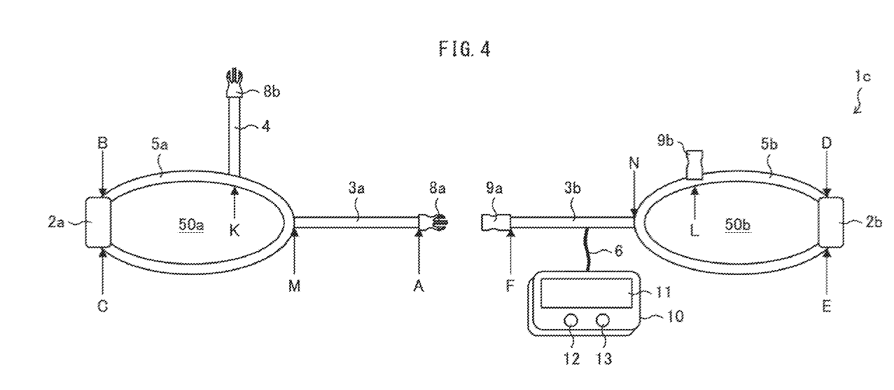

Next, a wearable instrument 1c will be described in reference to FIG. 4. The wearable instrument 1c has forelimb-insertion openings 50a and 50b through which the respective forelimbs of the dog are to be passed. FIG. 4 is a schematic diagram of an exemplary structure of the wearable instrument 1c in accordance with Embodiment 2. Members of the present embodiment that have the same function as members of the previous embodiment are indicated by the same reference numerals, and description thereof is omitted.

The wearable instrument 1c includes a pull-up strap 3a (first and second contact sections; first and second pull-up sections) that can form the forelimb-insertion opening 50a through which the left forelimb is to be passed and a pull-up strap 3b (first and second contact sections; first and second pull-up sections) that can form the forelimb-insertion opening 50b through which the right forelimb is to be passed. The pull-up straps 3a and 3b are connectable via the male and female ends 8a and 9a of a buckle.

The electrode 2a (measuring unit) is provided on portion BC (first contact section, second contact section) which provides a part of the periphery of the forelimb-insertion opening 50a and which comes into direct contact with one of the axillae of the dog. The electrode 2b (measuring unit) is provided on portion DE (first contact section, second contact section) which provides a part of the periphery of the forelimb-insertion opening 50b and which comes into direct contact with the other axilla of the dog. For example, if the left forelimb is passed through the forelimb-insertion opening 50a, the right forelimb is passed through the forelimb-insertion opening 50b, and the pull-up strap 3a and the pull-up strap 3b are connected on the back of the dog, the electrode 2a is pressed to the left axilla, and the electrode 2b is pressed to the right axilla. The cinch strap 4, arranged across the dog's anterior chest, then draws the pull-up strap 3a and the pull-up strap 3b together.

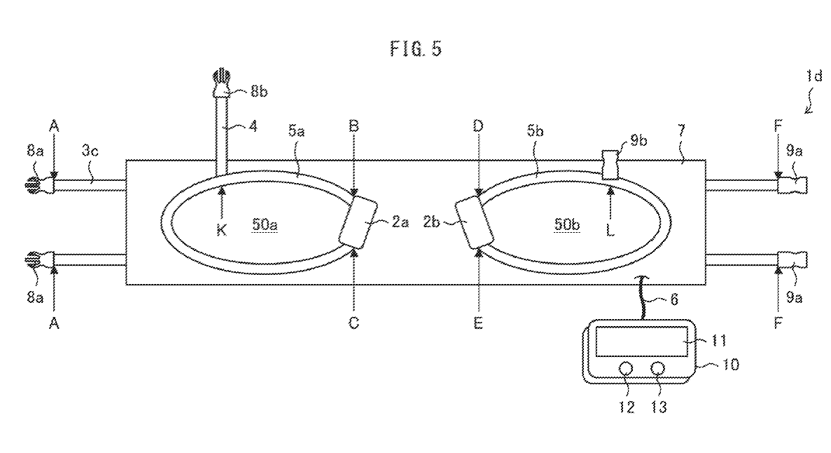

Alternatively, the forelimb-insertion openings 50a and 50b through which the forelimbs of the dog are passed may be arranged as in a wearable instrument 1d shown in FIG. 5. FIG. 5 is a schematic diagram of an exemplary structure of the wearable instrument 1d.

The wearable instrument 1d includes a cloth section 7 forming a part of pull-up straps 3c (first and second contact sections; first and second pull-up sections). The cloth section 7 has a forelimb-insertion opening 50a through which the left forelimb is to be passed and a forelimb-insertion opening 50b through which the right forelimb is to be passed. The electrode 2a is provided on portion BC which provides a part of the periphery of the forelimb-insertion opening 50a and which comes into direct contact with one of the axillae of the dog. The electrode 2b is provided on portion DE which provides a part of the periphery of the forelimb-insertion opening 50b and which comes into direct contact with the other axilla of the dog. The pull-up straps 3c include the male and female ends 8a and 9a of a buckle that are arranged to be connected on the back of the dog.

For example, if the left forelimb is passed through the forelimb-insertion opening 50a, the right forelimb is passed through the forelimb-insertion opening 50b, and the male and female buckle halves 8a and 9a of the pull-up straps 3c are connected on the dog's back, the electrode 2a is pressed to the left axilla, and the electrode 2b is pressed to the right axilla.

The electrodes 2a and 2b may be, for example, detachable from the cloth section 7 or the forelimb-insertion openings 50a and 50b. This arrangement allows for easy modification of the locations of the electrodes 2a and 2b to match the physical body size of the dog.

The cloth section 7 may be made of materials including those with suitable stretchiness and/or compressibility such as knitted fabric (e.g., jersey) and soft silicone. This arrangement makes the dog wearing the wearable instrument 1c feel less uncomfortable and enhances the snugness of the wearable instrument 1c when it is worn.

### Embodiment 3

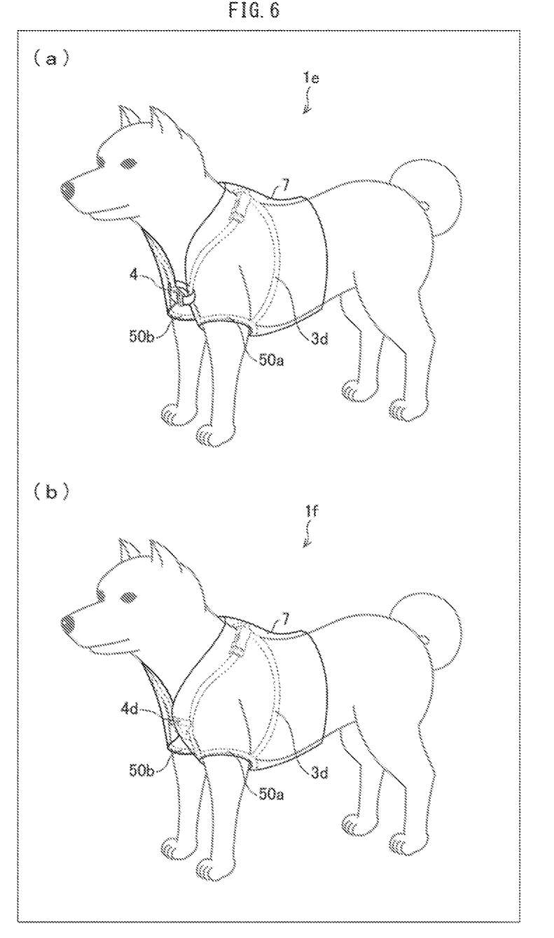

Another embodiment of the present invention will now be described in reference to FIG. 6. Portion (a) of FIG. 6 is a perspective view of a typical example of a dog wearing a wearable instrument 1e in accordance with Embodiment 3, and portion (b) of FIG. 6 is a perspective view of a typical example of a dog wearing a wearable instrument 1f in accordance with Embodiment 3. Members of the present embodiment that have the same function as members of any of the previous embodiments are indicated by the same reference numerals for convenience of description, and description thereof is omitted.

Both the wearable instruments 1e and 1f are shaped like a piece of clothing having forelimb-insertion openings 50a and 50b through which the forelimbs of the dog are to be passed. The electrodes 2a and 2b (measuring units), which will be placed under the axillae, are provided respectively on the peripheries of the forelimb-insertion openings 50a and 50b that will come into direct contact with the dog's axillae. Both the wearable instruments 1e and 1f include a pull-up mechanism 3d (first and second contact sections; first and second pull-up sections) (broken line) and a cloth section 7 that is shaped like a piece of clothing either on or under the pull-up mechanism 3d. The pull-up mechanism 3d pulls up toward the back of the dog those parts (first and second contact sections) of the peripheries of the forelimb-insertion openings 50a and 50b which come into direct contact with the axillae, so as not to allow for displacement of the electrodes 2a and 2b.

The pull-up mechanism 3d may be a string, a belt, or a rubber band. The cloth section 7 may be provided with a guiding unit like belt loops (not shown) such that the pull-up mechanism 3d is passed through the guiding unit. Alternatively, the cloth section 7 may be knitted in a manner that changes stepwise as to generate a force pulling up those parts of the cloth section 7 which come into direct contact with the axillae toward the dorsal region. In such a case, the cloth section 7 and the pull-up mechanism 3d are provided as a single piece.

The wearable instrument 1e shown in (a) of FIG. 6 includes the cinch strap 4 (cinch section) (broken line) that draws the left and right front portions of the pull-up mechanism 3d together across the dog's anterior chest. The wearable instrument 1f shown in (b) of FIG. 6 includes a cinch mechanism 4d (cinch section) (broken line) that draws the left and right front portions of the pull-up mechanism 3d together across the dog's anterior chest. Similarly to the pull-up mechanism 3d, the cinch mechanism 4d may be a string, a belt, or a rubber band or may be provided as an integral part of the cloth section 7 by knitting the cloth section 7 in such a manner that changes stepwise.

The cloth section 7 may be provided so as to cover the dog's whole body or to be worn on a part of the dog's body such as the upper body or the dorsal region.

The pull-up mechanism 3d is not necessarily provided as the peripheries of the forelimb-insertion openings 50a and 50b or otherwise shaped similarly to circles through which the dog's forelimbs are to be passed. For example, the pull-up mechanism 3d may be partly shaped like a string similarly to the pull-up strap 3 shown in FIG. 1. For example, the wearable instrument 1f may include: the cloth section 7 structured to be worn over the dog's dorsal region and shoulders; and the cinch mechanism 4d to be placed across the dog's anterior chest to draw the single left and right stretches of the pull-up mechanism 3d together, the string-like pull-up mechanism 3d being provided on the cloth section 7 at such locations that the two stretches come into direct contact with the dog's left and right shoulders when the wearable instrument 1f is worn by a dog. The wearable instrument 1f, when arranged in this manner, can be attached to a dog by passing the string-shaped pull-up mechanism 3d under the dog's axillae and pulling up toward the back of the dog those parts of the pull-up mechanism 3d which are in direct contact with the axillae. Alternatively, the wearable instrument 1f may be arranged such that the cloth section 7 covers the pull-up mechanism 3d and the cinch mechanism 4d when the wearable instrument 1f is worn by a dog.

### Variation Examples

The electrocardiograph main body 10 and the connecting cable 6 could disrupt movements of the dog when the wearable instrument 1 and 1a to 1f is attached to a dog. To avoid this from happening, the electrocardiograph main body 10 and the connecting cable 6 may be put in a storage bag 15 (rucksack) carried by the dog on its back.

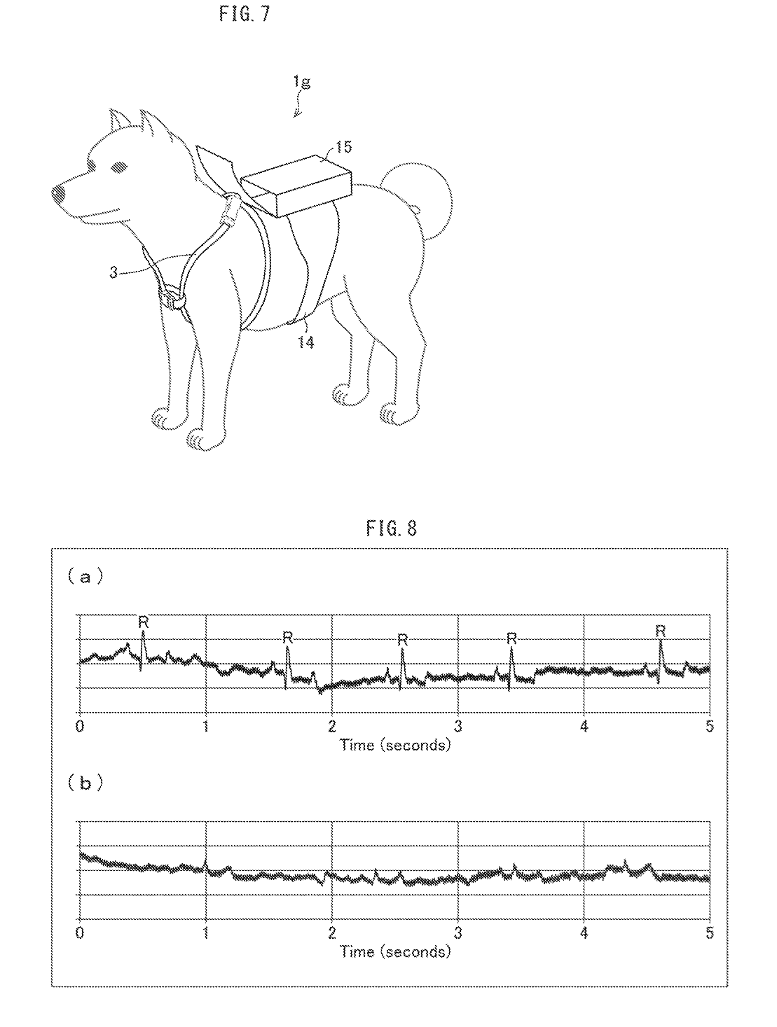

Here, a wearable instrument 1g, which is a combination of the wearable instrument 1 and the storage bag 15 (rucksack), will be described in reference to FIG. 7. FIG. 7 is a perspective view of an example of the exterior of the storage bag 15 in which the electrocardiograph main body 10 is contained.

A strap 14 for enabling the dog to carry the storage bag 15 on its back does not need to be fastened around the dog's trunk so tightly as to firmly secure the storage bag 15 onto the dog. It is desirable however that the wearable instrument 1g be sufficiently secured such that the pull-up strap 3 of the wearable instrument 1g is not displaced by the weight of, for example, the electrocardiograph main body 10 contained in the storage bag 15.

For example, the storage bag 15 may be provided on its back with a belt loop through which the pull-up strap 3 of the wearable instrument 1g is to be passed.

If the electrocardiograph main body 10 and the connecting cable 6 are sufficiently compact and lightweight, the storage bag 15 may be provided anywhere on the wearable instrument 1g or attached to a collar or like accessory to be worn by a dog.

As an alternative, the electrocardiograph main body 10 may be provided as a stand-alone device if the electrocardiograph main body 10 is of a large size. In this case, the electrocardiograph main body 10 may be placed near the cage of the dog and electrically connected to the wearable instrument 1g via the connecting cable 6.

### Embodiment 4

Portion (a) of FIG. 8 is a diagram representing an exemplary electrocardiographic waveform recorded by the wearable instrument 1 of Embodiment 1 worn by a dog, and (b) of FIG. 8 is a diagram representing an exemplary waveform recorded when either one of the electrodes 2a and 2b (measuring units) is not correctly pressed onto an axilla of the dog.

An action current caused by excitation of heart muscle is picked up by the electrodes 2a and 2b and recorded as an electrocardiographic waveform in which, for example, hum noise is superimposed on a base line as shown in (a) of FIG. 8.

A typical electrocardiogram is composed of P, Q, R, S, and T waves. All these P, Q, R, S, and T waves may appear distinctly or at least one of the waves may appear clearly and repeatedly in the electrocardiogram recorded using the electrodes 2a and 2b. Portion (a) of FIG. 8 shows an example where one of the P, Q, R, S, and T waves, namely the R wave, is measured clearly and repeatedly (approximately once every second).

One can measure a heart rate and identify normal/abnormal excitation of the ventricles and atria from the electrocardiogram shown in (a) of FIG. 8. In contrast, if the action current caused by excitation of heart muscle is not well measured as in (b) of FIG. 8, there appears no significant difference between the amplitude of hum noise on the base line and the amplitude of a wave that corresponds to the action current, which indicates that either the electrode 2a or 2b is not correctly pressed onto an axilla.

The electrocardiograph main body 10 may include a decision mechanism for determining whether or not either the electrode 2a or 2b is correctly pressed onto an axilla and a notification unit (e.g., a red/blue lamp or a sound output unit) for notifying the animal owner of a determination. For example, the electrocardiograph main body 10 may turn on a red lamp, indicating an error, if a waveform like the one shown in (b) of FIG. 8 is being observed and turn on a blue lamp, indicating normal operation, if a waveform like the one shown in (a) of FIG. 8 is being observed. It will also be appreciated that the electrocardiograph main body 10 may communicate with an external display (e.g., mobile phone) so that the external display can display a determination.

The decision mechanism for determining whether or not either the electrode 2a or 2b is incorrectly pressed onto an axilla may do so based on calculation of an S/N ratio, Fourier conversion, or evaluation of an auto-correlation function.

The electrocardiograph main body 10 may include, in this manner, a decision mechanism and a notification unit, the decision mechanism determining whether or not the electrodes 2a and 2b are pressed correctly onto the axillae. This arrangement enables a quick notification to the animal caretaker that the dog's heart rate is not being measured correctly, thereby urging the dog's caretaker to re-attach the electrodes 2a and 2b. Such a decision mechanism achieves similar effects when it is used in measurement of body temperature, pulse wave, amount of perspiration, respiration rate, and body fat.

### Embodiment 5

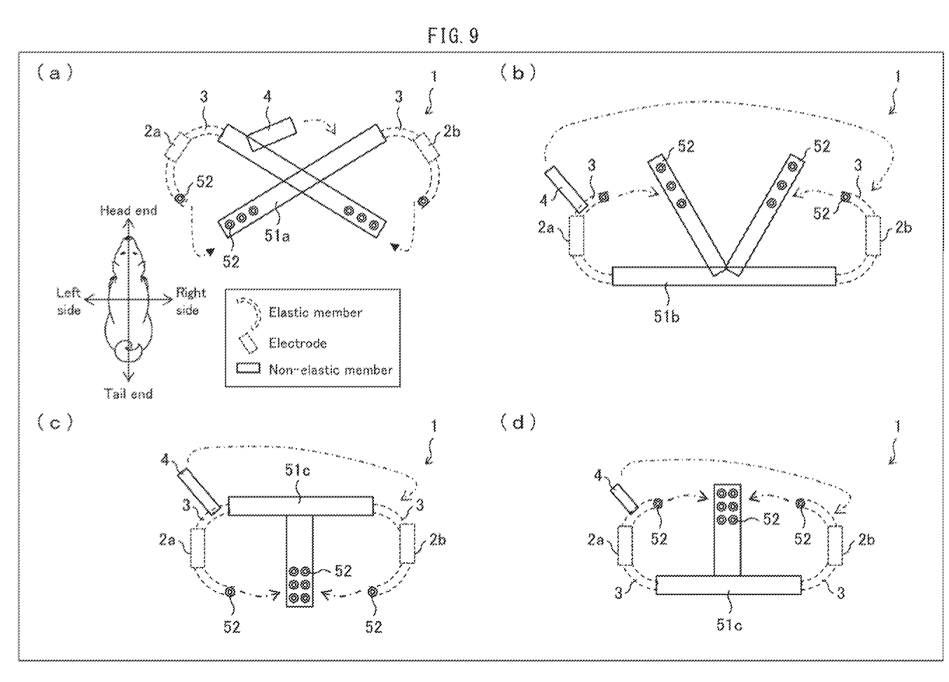

FIG. 9 is schematic views of other exemplary shapes of the wearable instrument 1 in accordance with another aspect of the present invention. The wearable instruments 1 shown in (a) to (d) of FIG. 9 include portions made of non-elastic members 51a, 51b, and 51c (e.g., synthetic leather or cloth). In the example shown in the figure, the pull-up strap 3 and the electrodes 2a and 2b are connected to the non-elastic members 51a, 51b, and 51c. As is the case with the previous examples, the pull-up strap 3 has sections that come into direct contact with the dog's axillae, and the electrodes 2a and 2b are provided on these sections. Meanwhile, the cinch strap 4 draws the left and right front portions of the pull-up strap 3 together across the dog's anterior chest.

To attach the wearable instrument 1 shown in (a) to (d) of FIG. 9 to a dog, first, the non-elastic member 51a, 51b, or 51c is attached to the dog over the back of the dog in such a manner as to spread it over the dog. Next, the left and right portions of the pull-up strap 3 (including the electrodes 2a and 2b) hanging from the shoulders of the dog are passed under the respective left and right forelimbs of the dog. The pull-up strap 3 is then connected to the non-elastic member 51 using a button 52 on the pull-up strap 3 and a button 52 on the non-elastic member 51a, 51b, or 51c.

The solid lines in FIG. 9 indicate the non-elastic members 51a, 51b, and 51c, the long broken lines indicate an elastic member (e.g., the pull-up strap 3), and the short broken lines indicate electrodes. The dash-dot lines in FIG. 9 indicate relationships between sites to be connected using the buttons 52 or a buckle (not shown).

The wearable instrument 1 shown in FIG. 9 is built such that the cinch strap 4 is integral to the non-elastic member 51a, 51b, or 51c or the pull-up strap 3, which is illustrative only. The buttons 52 are shown in the figure and used as an example to connect an end of the pull-up strap 3 to the non-elastic member 51a, 51b, or 51c, which by no means limits the present embodiments. Alternatively, the buttons 52 may be replaced by press studs, hook and loop fasteners, or a buckle.

Portion (a) of FIG. 9 shows an example where the non-elastic members 51a are arranged like a letter “X” on the back of the dog (in direct contact with the back). The cinch strap 4 is placed before the non-elastic members 51a. The wearable instrument 1 shown in (a) of FIG. 9, when attached to a dog, appears like the wearable instrument 1 shown in (c) of FIG. 2. More specifically, the non-elastic members 51a, when worn, go on the back of the dog, and the pull-up strap 3 made of an elastic member goes under and around the axillae.

Portion (b) of FIG. 9 shows an example where the non-elastic member 51b is arranged like a letter “K” on the back of the dog (in direct contact with the back). The cinch strap 4 is placed before the pull-up strap 3. This arrangement prevents the wearable instrument 1 from spreading over and near the dog's shoulder joints when the wearable instrument 1 is worn by the dog. The arrangement hence minimizes interference of the wearable instrument 1 with the dog's shoulder blades when the wearable instrument 1 is worn by a dog. That in turn prevents the wearable instrument 1 from being displaced by the movement of the shoulder blades that inevitably occurs when the dog moves. This arrangement of the wearable instrument 1 is suited, for example, for relatively lean dogs, especially for dogs with such a physical structure that the movement of the shoulder blades tends to reach the skin of the dog.

Portion (c) of FIG. 9 shows an example where the non-elastic member 51c is arranged like a letter “T” on the back of the dog. Meanwhile, (d) of FIG. 9 shows an example where the non-elastic member 51c is arranged like an inverted letter “T” on the back of the dog. In both the examples shown in (c) and (d) of FIG. 9, the cinch strap 4 is placed before the pull-up strap 3. This arrangement also minimizes interference of the wearable instrument 1 with the dog's shoulder blades similarly to the example in (b) of FIG. 9. The non-elastic member 51c may also be arranged like a letter “H” on its side, to take advantage of the features of both (c) and (d) of FIG. 9 (not shown). FIG. 9 gives examples of letter-like shapes, which is illustrative only. Alternatively, for example, the non-elastic members 51a, 51b, and 51c may be shape like a letter “Y.”

The arrangements of the wearable instrument 1 shown in (a) to (d) of FIG. 9 allow the wearable instrument 1 to either partly (e.g., where those parts which come in direct contact with the axillae) or entirely fit the dog's body snugly. Accordingly, the arrangements enable the wearable instrument 1 to be worn by the dog in such a manner that the wearable instrument 1 stays snugly fitting the dog's body surface in any posture of the dog. Thus, the wearable instrument 1 is capable of measurement of various biological information without being substantially affected by the dog's motion or posture. LED-based light-receiving/emitting units, thermopiles, and various other sensors may be provided, replacing the electrodes 2a and 2b, in the non-elastic member 51 or on the pull-up strap 3 of the wearable instrument 1 for stable measurement of various biological information.

### Overview

The present invention, in aspect 1 thereof, is directed to a biological information measuring instrument (wearable instrument 1 and 1a to 1g) to be worn by an animal for measurement of biological information of the animal, the instrument including: at least one measuring unit (electrode 2a, 2b) to be placed under at least one of left and right axillae of the animal to measure the biological information; a first contact section (pull-up strap 3, 3a to 3c; pull-up mechanism 3d) to be placed in direct contact with one of the left and right axillae; a second contact section (pull-up strap 3, 3a to 3c; pull-up mechanism 3d) to be placed in direct contact with the other one of the left and right axillae; a first pull-up section (pull-up strap 3, 3a to 3c; pull-up mechanism 3d) configured to pull up the first contact section toward a dorsal region of the animal; a second pull-up section (pull-up strap 3, 3a to 3c; pull-up mechanism 3d) configured to pull up the second contact section toward the dorsal region; and a cinch section (cinch strap 4; cinch mechanism 4d) to be placed across an anterior chest of the animal to draw the first and second pull-up sections together, wherein at least one of the first and second contact sections is configured to press the measuring unit to the associated one of the axillae.

According to this aspect of the invention, the first and second pull-up sections pull up the first and second contact sections respectively toward the dorsal region of the animal, thereby pressing the measuring unit(s) provided for at least one of the left and right axillae to that axilla. Furthermore, the cinch section draws the first and second pull-up sections together across the anterior chest of the animal, thereby maintaining the first pull-up section pressing the measuring unit to the associated axilla and also maintaining the first and second pull-up sections in place. The aspect therefore stably maintains the measuring unit pressed to an axilla of the animal.

In aspect 2 of the present invention, the biological information measuring instrument of aspect 1 may be configured such that the at least one measuring unit includes at least one measuring unit to be placed under each of the left and right axillae of the animal.

According to this aspect of the invention, the first and second pull-up sections pull up the axillae toward the dorsal region of the animal such that the measuring units are pressed to the axillae of the animal. For example, the electrodes for recording electrocardiographic data are placed under the left and right axillae. The aspect is applicable to the measuring units pressed to the left and right axillae in the same manner as it is applicable to the electrodes for recording electrocardiographic data.

In aspect 3 of the present invention, the biological information measuring instrument of aspect 2 may be configured such that the measuring units each include an electrode connected to an electrocardiograph (electrocardiograph main body 10).

According to this aspect of the invention, the electrodes are pressed to the left and right axillae while electrocardiographic data is recorded.

In aspect 4 of the present invention, the biological information measuring instrument of any one of aspects 1 to 3 may be configured to be shaped like a strap so as to be attached onto a body surface of the animal.

According to this aspect of the invention, the biological information measuring instrument has a simple structure.

In aspect 5 of the present invention, the biological information measuring instrument of any one of aspects 1 to 3 may be configured such that: the biological information measuring instrument is shaped like a piece of clothing with forelimb-insertion openings through which respective forelimbs of the animal are to be passed and the first and second contact sections each include a portion to be placed in direct contact with the associated one of the axillae, the portion forming a periphery of the associated one of the forelimb-insertion openings.

According to this aspect of the invention, at least one measuring unit is provided either on the periphery of the forelimb-insertion opening in the first contact section or on the periphery of the forelimb-insertion opening in the second contact section. That arrangement in turn presses the measuring unit to the associated one of the axillae.

In aspect 6 of the present invention, the biological information measuring instrument of any one of aspects 1 to 5 may be configured to further include a biological information processing unit (electrocardiograph main body 10) configured to obtain measurements produced by the measuring unit and generate output data containing the biological information of the animal from the measurements.

The present invention in various aspects thereof is not limited to the description of the embodiments above, but may be altered by a skilled person within the scope of the claims. An embodiment based on a proper combination of technical means disclosed in different embodiments is encompassed in the technical scope of the present invention. Furthermore, a new technological feature may be created by combining different technological means disclosed in the embodiments.

### CROSS-REFERENCE TO RELATED APPLICATIONS

The present application hereby claims priority to Japanese Patent Application, Tokugan, No. 2015-157586 filed Aug. 7, 2015, the entire contents of which are hereby incorporated herein by reference.

### INDUSTRIAL APPLICABILITY

The present invention, in one aspect thereof, can be used in attaching a biological information measurement tool to a dog or like animal.

### REFERENCE SIGNS LIST

1, 1a to 1g Wearable Instrument (Biological Information Measuring Instrument)
2a, 2b Electrode (Measuring Unit)
3, 3a to 3c Pull-up Strap (First and Second Contact Sections, First and Second Pull-up Sections)
3d Pull-up Mechanism (First and Second Contact Sections, First and Second Pull-up Sections)
4 Cinch Strap (Cinch Section)
4d Cinch Mechanism (Cinch Section)
10 Electrocardiograph Main Body (Electrocardiograph, Biological Information Processing Unit)
50A, 50b Forelimb-insertion Opening

1. A biological information measuring instrument to be worn by an animal for measurement of biological information of the animal, the instrument comprising:
a first contact section to be placed in direct contact with the left axillae of the animal;
a second contact section to be placed in direct contact with the right axillae of the animal;
at least one electrode provided on at least one of the first contact section and the second contact section;
a first pull-up section configured to pull up the first contact section toward a dorsal region of the animal;
a second pull-up section configured to pull up the second contact section toward the dorsal region; and
a cinch section to be placed across an anterior chest of the animal to draw the first and second pull-up sections together,
wherein at least one of the first and second contact sections is configured to press the at least one electrode to the axillae of the animal.

2. The biological information measuring instrument according to claim 1, wherein the at least one electrode comprises at least one electrode to be placed under each of the left and right axillae of the animal.

3. The biological information measuring instrument according to claim 2, wherein the electrode is connected to an electrocardiograph.

4. The biological information measuring instrument according to claim 1, shaped like a strap so as to be attached onto a body surface of the animal.

5. The biological information measuring instrument according to claim 1, further comprising
forelimb-insertion openings through which respective forelimbs of the animal are to be passed; wherein
the first and second contact sections each comprise a portion to be placed in direct contact with the associated one of the axillae, the portion forming a periphery of the associated one of the forelimb-insertion openings.

6. The biological information measuring instrument according to claim 1, further comprising a biological information processing unit configured to obtain measurements produced by the at least one electrode and generate output data containing the biological information of the animal from the measurements.

7. The biological information measuring instrument according to claim 1, further comprising,
an electrocardiograph connected with the at least one electrode via a connecting cable containing an electrically conductive substance, wherein
the connecting cable provided in the first pull-up section or the second pull-up section.

8. The biological information measuring instrument according to claim 5, wherein
the forelimb-insertion openings comprises a first forelimb-insertion opening and a second forelimb-insertion opening,
the first contact section forms a periphery of the first forelimb-insertion openings, and
the second contact section forms a periphery of the second forelimb-insertion openings.

9. The biological information measuring instrument according to claim 8, further comprising,
a cloth section including the first contact section, the second contact section, the cinch section, the first forelimb-insertion opening and the second forelimb-insertion opening.

10. The biological information measuring instrument according to claim 1, wherein
the first pull-up section and the second pull-up section are shaped like a strap, and
the first pull-up section comprises a first fastening member, and the second pull-up section comprises a second fastening member and
the first fastening member is connectable to the second fastening member.

11. The biological information measuring instrument according to claim 10, wherein
the first fastening member is adjustable in a length of the first pull-up section or the second fastening member is adjustable in a length of the second pull-up section.
